(19) United States
(12) Patent Application Publication
(10) Pub. No.: US 2007/0202143 A1
(43) Pub. Date: Aug. 30, 2007

(54) Direct application of non-toxic crosslinking reagents to restabilize surgically destabilized intervertebral joints

(76) Inventor: Thomas P. Hedman, Stevenson Ranch, CA (US)

Correspondence Address: Robert Berliner;BERLINER & ASSOCIATES, 31st Floor, 555 W. Fifth Street, Los Angeles, CA, 90013, US

(21) Appl. No.: 11/726,790

(22) Filed: Mar. 22, 2007

Related U.S. Application Data
(63) Continuation-in-part of application No. 11/712,684, filed on Feb. 28, 2007.
Application No. 11/712,684 is a continuation-in-part of application No. 11/346,464, filed on Feb. 2, 2006.
Application No. 11/346,464 is a continuation-in-part of application No. 10/786,861, filed on Feb. 24, 2004.
Application No. 11/346,464 is a continuation-in-part of application No. 10/230,671, filed on Aug. 29, 2002.
(60) Provisional application No. 60/498,790, filed on Aug. 28, 2003.
(60) Provisional application No. 60/316,287, filed on Aug. 31, 2001.

(51) Int. Cl. A61K 9/00

(52) U.S. Cl. 424/422

(57) ABSTRACT

A method of improving the resistance of collagenous tissue subject to elevated collagenous tissue stress as a result of tissue removing surgical decompression surgery, comprising contacting at least a portion of the remaining collagenous tissue with an effective amount of a crosslinking reagent.

### CROSS-REFERENCE TO RELATED APPLICATIONS

This application is a continuation-in-part of application Ser. No. 11/712,684, filed on Feb. 28, 2007, which is a continuation-in-part of application Ser. No. 11/346,464, filed on Feb. 2, 2006, which is a continuation-in-part of application Ser. No. 10/786,861, filed on Feb. 24, 2004, which claims the benefit of U.S. Provisional Application Ser. No. 60/498,790, filed on Aug. 28, 2002, and which is a continuation-in-part of application Ser. No. 10/230,671, filed on Aug. 29, 2002, which claims the benefit of U.S. Provisional Application Ser. No. 60/316,287, filed on Aug. 31, 2001.

### BACKGROUND OF THE INVENTION

1. Field of the Invention

The present invention relates to a method for treatment of tissue, for example, collagenous tissue, where surgical removal or ablation of the collagenous tissue or of adjacent tissues has produced a deleterious mechanical loading environment which contributes to the degradation of the tissue.

2. Description of the Related Art

Deleterious mechanical loading environments contribute to the degradation of collagenous tissue in a variety of manners. For instance, fatigue is a weakening of a material due to repetitive applied stress. Fatigue failure is simply a failure where repetitive stresses have weakened a material such that it fails below the original ultimate stress level. Elevated stress levels, due to tissue removal, can accelerate fatigue degradation of the remaining joint tissues. In bone and other diarthrodial joint tissues, two processes—biological repair and fatigue—are in opposition, and repair generally dominates. In the intervertebral disc, the prevalence of mechanical degradation of the posterior annulus (Osti 1992) suggests that fatigue is the dominant process. The intervertebral disc, being the largest, principally avascular load supporting tissue in the body, is somewhat unique in this predisposition toward ongoing fatigue degradation. Active tissue response (adaptation, repair) does not play a strong role in the case of mature intervertebral disc material. The intervertebral disc is comprised of three parts: the nucleus pulposus (NP) or nucleus, the annulus fibrosus (AF) or annulus, and the cartilaginous endplates. The characteristic of the inner annulus and outer nucleus blend with ongoing degeneration, with the nucleus becoming more fibrous and decreasing in water content. Similarly, the boundary between outer nucleus and inner annulus is known to fade and becomes indistinct with ongoing degeneration. As a principally avascular structure, the disc relies on diffusion and loading induced convection for nutrition of its limited number of viable cells. Age related changes interfere with diffusion presumably contributing to declining cell viability and biosynthetic function (Buckwalter et al. 1993, Buckwalter 1995). Age related decline in numbers of cells and cell functionality compromises the ability of the cells to repair mechanical damage to the matrix. Some regeneration of the matrix in the nucleus following enzymatic degradation has been accomplished, albeit inconsistently (Deutman 1992). Regeneration of functional annular material has not yet been realized.

Combined with this limited potential for repair or regeneration, studies have shown that posterior intervertebral disc tissue is vulnerable to degradation and fatigue failure when subjected to non-traumatic, physiologic cyclic loads. Prior work has shown deterioration in elastic-plastic (Hedman 99) and viscoelastic (Hedman 02) material properties in posterior intervertebral disc tissue subjected to moderate physiological cyclic loading. Cyclic load magnitudes of 30% of ultimate tensile strength produced significant deterioration of material properties with as little as 2000 cycles. Green (1993) investigated the ultimate tensile strength and fatigue life of matched pairs of outer annulus specimens. They found that fatigue failure could occur in less than 10,000 cycles when the vertical tensile cyclic peak exceeded 45% of the ultimate tensile stress of the matched pair control. In addition, Panjabi et al (1996) found that single cycle sub-failure strains to anterior cruciate ligaments of the knee alter the elastic characteristics (load-deformation) of the ligament. Osti (1992) found that annular tears and fissures were predominantly found in the posterolateral regions of the discs. Adams (1982) demonstrated the propensity of slightly degenerated discs to prolapse posteriorly when hyperflexed and showed that fatigue failure might occur in lumbar discs as the outer posterior annulus is overstretched in the vertical direction while severely loaded in flexion. In an analytical study, interlaminar shear stresses, which can produce delaminations, have been found to be highest in the posterolateral regions of the disc (Goel 1995). These prior data indicate: 1) the posterior disc and posterior longitudinal ligament are at risk of degenerative changes, and that 2) the mechanism of degeneration can involve flexion fatigue.

Stress intensification due to tissue removal can be expected to decrease fatigue resistance in the joint tissues, leading to accelerated degradation. An example of this type of accelerated joint tissue degradation is the mechanical degradation of collagenous tissue which occurs subsequent to spinal decompression surgery. Progressive spinal degradation can occur subsequent to surgical bone removal, with or without removal of part of the intervertebral disc as is done in a discectomy procedure. With surgical removal of bone, disc and other connective tissues, the spinal segment can have elevated tissue stresses due to normal physiologic loading. Discectomy procedures, in particular, have been shown to increase the neutral zone, a common parameter used to quantify the degree of spinal joint instability (Chuang and Hedman 2007). Spinal joint instability is thought to lead to accelerated tissue degeneration and clinical symptoms.

Naturally occurring collagen crosslinks play an important role in stabilizing collagenous tissues and, in particular, the intervertebral disc. Significantly higher quantities of reducible (newly formed) crosslinks have been found on the convex sides than on the concave sides of scoliotic discs (Duance, et al. 1998). Similarly, Greve, et al. (1988) found a statistically increased amount of reducible crosslinks in scoliotic chicken discs at the same time that curvatures were increasing. This suggests that there is some form of natural, cell-mediated crosslink augmentation that occurs in response to the elevated tensile environment on the convex side of scoliotic discs. Greve also found that there were fewer reducible crosslinks at the very early stages of development in the cartilage of scoliotic chickens. They concluded that differences in collagen crosslinking did not appear to be causative because there was not a smaller number of crosslinks at later stages of development. In fact, later on, when the scoliotic curve was progressing, there were statistically significant greater numbers of collagen crosslinks, perhaps in response to the curvature. Although not the conclusion of Greve, this can be interpreted as being a sufficient depletion of crosslinks in the developmental process with long enough duration to trigger the progression of scoliotic curvature that was later mended by a cellular response that produced higher than normal levels of crosslinks. These studies suggest that the presence of collagen crosslink augmentation mechanisms may be critical to prevent ongoing degradation and for mechanical stability of intervertebral disc tissue in scoliotic spines and when tensile stresses are elevated.

It is well documented that endogenous (naturally occurring—enzymatically derived and age increasing non-enzymatic) and exogenous collagen crosslinks (historically applied to implants) increase the strength and stiffness of collagenous, load-supporting tissues (Chachra 1996, Wang 1998, Sung 1999a, Zeeman 1999, Chen 2001). Sung (1999b) found that a naturally occurring crosslinking agent, genipin, provided greater ultimate tensile strength and toughness when compared with other crosslinking reagents. Genipin also demonstrated significantly less cytotoxicity compared to other more commonly used crosslinking agents. With regard to viscoelastic properties, Lee (1989) found that aldehyde fixation reduced stress-relaxation and creep in bovine pericardium. Recently, naturally occurring collagen crosslinks were described as providing ‘sacrificial bonds’ that both protect tissue and dissipate energy (Thompson, et al. 2001). There is no known reference in the literature as to the ability of directly applied, exogenous collagen crosslink augmentation to restabilize surgically destabilized intervertebral joints. Joint stability is generally considered a complex phenomenon dependent on the elastic-plastic and viscoelastic mechanical properties of all involved joint tissues. For example, arthritic degradation may follow excessive stiffness or inadequate stiffness of a joint. Likewise, changes in the viscoelastic, time-dependent material properties of joint tissues could affect the types of stresses in the tissues leading to tissue degradation. Replication of normal, healthy joint mechanics is usually considered the goal of joint stabilization. Consequently, the preferred range of joint mechanical properties must usually be determined using experimental data. Joint mechanical property changes could arise due to joint trauma, tissue fatigue, or surgical intervention. The effects of degradative changes are heightened in tissues with limited capacity for biologic repair, such as in the avascular and nutritionally challenged intervertebral disc or the knee meniscus. While the overall success rate of lumbar discectomy is favorable, especially regarding immediate pain relief and return to work, biomechanical investigation (Goel, 1985, 1986) and long-term clinical results (Kotilainen, 1993, 1994, 1998) suggest altered kinematic behavior and degenerative changes to the lumbar spine associated with significant loss of nucleus material and disc height, including the potential for lumbar instability. Currently, no treatments are available to aide in the prevention of instability and the subsequent degeneration following disc surgery. A need therefore exists for a treatment that can prevent spinal degeneration by restoring some of the inherent stability of the intervertebral joint subsequent to surgical decompression surgeries

### SUMMARY OF THE INVENTION

The present invention overcomes the deficiencies of the prior art in providing biochemical methods including collagen crosslink augmentation to prevent spinal degeneration by restoring some of the inherent stability of the intervertebral joint subsequent to tissue removal surgical decompression surgeries.

It is one object of the present invention to provide a method curtailing the progressive mechanical degradation of intervertebral disc tissue subsequent to tissue removing surgical decompression by increasing crosslinks in the collagenous tissues above native, intrinsic levels while otherwise maintaining the intrinsic characteristics of the treated tissues.

It is another object of the present invention to provide such a method that uses crosslinking reagents with substantially less cytotoxicity compared to common aldehyde fixation agents in order to facilitate direct contact of these reagents to tissues in the living human body.

It is another object of the present invention to increase such crosslinking of disc annular tissue by directly contacting living human disc tissue with appropriate concentrations of a non-toxic crosslinking reagent (or a mixture of crosslinking reagents) such as genipin (a geniposide) or proanthrocyanidin (a bioflavonoid) or Methylglyoxal, or threose, or EDC, or transglutaminase, or lysyl oxidase.

It is another object of the present invention to increase such crosslinking with a treatment method for minimally invasive delivery of the non-cytotoxic crosslinking reagent such as injections directly into the select tissue using a needle, for example into the remaining disc subsequent to a discectomy procedure, or placement of a time-release delivery system such as a carrier gel or ointment, or a treated membrane or patch directly into or onto the target tissue.

In accordance with the present invention, there is provided a method for treatment that is applied subsequent to or in combination with tissue removing surgical decompression to improve fatigue resistance and joint stability using non-toxic crosslinking compositions that are effective fatigue inhibitors and intervertebral joint stabilizers.

A method of the present invention comprises the step subsequent to or in combination with tissue removing surgical decompression of contacting at least a portion of remaining collagenous tissue with an effective amount of a crosslinking reagent. The crosslinking reagent includes a crosslinking agent such as genipin and/or proanthrocyanidin and/or EDC and/or a sugar such as ribose or threose, and/or byproducts of metabolism and advanced glycation end products (AGEs) such as glyoxal or methylglyoxyl and/or an enzyme such as lysyl oxidase (LO) enzyme (either in purified form or recombinant), or transglutaminase (Tgase), and/or a LO or Tgase promoter, and/or an epoxy or a carbodiimide. Preferably, the crosslinking reagent contains at least 100 mM methylglyoxal and/or 0.25% genipin. More preferable is a crosslinking reagent with a concentration of 400 mM methylglyoxal and/or 0.33% genipin. Further, the crosslinking reagent may include a crosslinking agent in a carrier medium. Preferably, the crosslinking reagent contains one of the following ranges of agent concentrations or a combination of agent concentrations: at least 0.001% (0.01 mg/ml) of human recombinant transglutaminase, at least 0.01% (0.1 mg/ml) of purified animal liver transglutaminase, at least 0.25% genipin, at least 0.1% proanthrocyanidin, at least 100 mM EDC, at least 100 mM ribose, at least 100 mM L-Threose, at least 50 mM methylglyoxal, at least 50 mM glyoxal, at least 0.001% lysyl oxidase in a 0.1 M urea solution. Further, the crosslinking reagent may include a crosslinking agent in a carrier medium.

The collagenous tissue to be contacted with the crosslinking reagent is a portion of an intervertebral disc remaining after tissue removing surgical decompression. The contact between the tissue and the crosslinking reagent is effected by injections directly into the select tissue using a needle. Alternatively, contact between the tissue and the crosslinking reagent is effected by placement of a time-release delivery system such as a gel or ointment, or a treated membrane or patch directly into or onto the target tissue. Contact may also be effected by, for instance, soaking or spraying.

It is another object of the present invention to provide biochemical methods that enhance the body's own efforts to stabilize spinal discs following tissue removing surgical decompression, by increasing collagen crosslinks.

It is another object of the present invention to cause this stability enhancement by reducing the bending hysteresis (energy lost in a complete loading-unloading cycle) and neutral zone size (the rotational range of the low stiffness region of the bending curve) and range of motion to normal or pre-surgical levels, and by increasing the bending strain energy (bending energy stored and returned) and stiffness in the low stiffness region of intervertebral joints to normal or pre-surgical levels following tissue removing surgical decompression, that is increasing the “bounce-back” characteristics from an imposed bending moment by injecting non-toxic crosslinking reagents into the involved discs.

It is another object of the present invention to enhance stability such that bending hysteresis and neutral zone size and range of motion and bending strain energy and stiffness in the low stiffness region return to the intrinsic levels prior to surgical intervention by injecting non-toxic crosslinking reagents into the discs to be surgically altered by tissue removing surgical decompression.

The appropriate locations for injection may be determined using three-dimensional reconstructions of the affected tissues as is possible by one skilled in the art, and combining these reconstructions with an algorithm to recommend the optimum placement of these reagents so as to affect-the greatest possible protection against instability and tissue degradation. These three-dimensional depictions of preferred locations for crosslinker application may display or highlight the surgically removed or altered tissues, and may best be created with custom computer software that incorporates any type of medical images of the patient that are available, and may best be displayed on a computer driven display device such as a lap-top computer or a devoted device. Additional, guidable, arthroscopic types of devices may be used, or developed or modified, to facilitate application of the reagents to appropriate areas on the intervertebral discs or adjacent cartilaginous, bony, capsular or ligamentous tissues.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, methods, devices, and application trays particularly pointed out in the appended claims.

### DESCRIPTION OF THE FIGURES

FIG. 1 is a graph of relaxation test results of two-way ANOVA analysis;

FIG. 2 is a graph of hardness test results caused by G2 crosslinking treatment;

FIG. 3 is chart comparing instability parameters for spinal collagenous tissue that is intact, subject to discectomy or crosslinked with a non-enzymatic (methylglyoxyl) reagent; and

FIG. 4 is chart comparing instability parameters for spinal collagenous tissue that is intact, subject to discectomy or crosslinked with an organic (genipin) reagent.

### DETAILED DESCRIPTION OF THE INVENTION

The present invention provides methods and devices for improving the resistance of collagenous tissues in the human body, where surgical removal or ablation of the collagenous tissue or to adjacent tissues has produced a deleterious mechanical loading environment which contributes to the degradation of the tissue, comprising the step of contacting at least a portion of a collagenous tissue with an effective amount of a crosslinking reagent. In one embodiment of the present invention, the method of the present invention also provides a method of curtailing the progressive mechanical degradation of such surgically impacted intervertebral disc tissue, and of improving fatigue resistance and joint stability, by enhancing the body's own efforts to stabilize mechanically insufficient tissues by increasing collagen crosslinks. In this embodiment, the present invention also provides for specific formulations of crosslinking reagents with substantially less cytotoxicity compared to common aldehyde fixation agents in order to facilitate direct contact of these reagents to tissues in the living human body.

In a second embodiment of the present invention, methods and devices are provided for stabilization, improving the fatigue resistance and preventing the progressive degradation of intervertebral discs and surrounding tissues following or accompanying a destabilizing surgical procedure such as a neural decompression procedure such as a laminectomy or laminotomy or facetectomy or discectomy, by increasing collagen crosslinks. Examples of the latter are progressive degradation and the associated pain subsequent to a posterior bony decompression with a discectomy, and a percutaneous discectomy. While these procedures when done correctly are generally effective for immediate relief of symptoms, their destabilizing effects to the surgically altered intervertebral joint is well documented. The destabilization caused by surgical excision of musculoskeletal tissues can in many cases lead to arthritic degeneration and long term degradation of the associated joint and joint tissues, leading to subsequent manifestations of pain, radiculopathy and other clinical symptoms. The present invention will be used to prevent arthritic degeneration of the joint and joint tissues by reestablishing the appropriate levels of joint stability, ensuring appropriate, physiological levels of tissue stresses, deformations and motions, by increasing collagen crosslinks in the collagenous tissues of the surgically affected joint.

The crosslinking reagent of the present invention is not particularly limited. Any crosslinking reagent known to be substantially non-cytotoxic and to be an effective cross-linker of collagenous material may be used. The crosslinking reagent is required to be substantially non-cytotoxic in order to facilitate direct contact of the crosslinking agent to tissues in the living human body. Preferably, the crosslinking reagent exhibits substantially less cytotoxicity compared to common aldehyde fixation agents. More preferably, a non-cytotoxic crosslinking reagent is used.

Appropriate cytotoxicity testing will be used to verify the minimal cytotoxicity of candidate crosslinking reagents prior to use in humans. Tissue specific in vitro tests of cytotoxicity, of the standard form applied to mouse connective tissue (F895-84(2001)e1 Standard Test Method for Agar Diffusion Cell Culture Screening for Cytotoxicity), or Chinese Hamster Ovaries (ASTM E1262-88(1996) Standard Guide for Performance of the Chinese Hamster Ovary Cell/Hypoxanthine Guanine Phosphoribosyl Transferase Gene Mutation Assay) preferably utilizing cell lines from tissues approximating the fibrous and gelatinous tissues of the intervertebral disc, should be conducted to evaluate the level of toxicity of any specific combination of crosslinking reagents known to have minimal cytotoxicity. These in vitro tests should similarly be followed by in vivo animal tests prior to use in humans.

The crosslinking reagent includes at least one crosslinking agent. The crosslinking agent chosen in accordance with the present invention is an effective cross-linker of collagenous material. When used in a cross-linking reagent, an effective crosslinker is one that increases the number of crosslinks in the collagenous tissue when the crosslinker is brought into contact with a portion of the collagenous tissue. Elevated stress levels, due to tissue removal, can accelerate fatigue degradation of the remaining joint tissues. Surgical tissue removal, as in posterior spinal decompression procedures such as discectomies, can produce mechanical instability in the affected joint leading to accelerated degradation of joint tissues and clinical symptoms. Therefore, an effective crosslinker improves the fatigue resistance of the treated tissue, reduces material property degradation resulting from repetitive physiologic loading, or stabilizes the affected joint and joint tissues. Likewise, an effective crosslinker may reduce the decrease in elastic-plastic properties due to fatigue loading of the treated tissue. In one embodiment of the present invention, the crosslinking agent is genipin, a substantially non-toxic, naturally occurring crosslinking agent. Genipin is obtained from its parent compound, geniposide, which may be isolated from the fruits of Gardenia jasminoides. Genipin may be obtained commercially from Challenge Bioproducts Co., Ltd., 7 Alley 25, Lane 63, TzuChiang St. 404 Taichung Taiwan R.O.C., Tel 886-4-3600852. In another embodiment of the present invention, the crosslinking agent is a bioflavonoid, and more specifically, the bioflavonoid is proanthrocyanidin. A mixture containing proanthrocyanidin can be obtained as MegaNatural.™. Gold from Polyphenolics, Inc, 22004 Rd. 24, Medera, Calif. 93638, Tel 559-637-5961. More than one crosslinking agent may be used. Appropriate cross-linking reagents will also include a sugar such as ribose or threose, or byproducts of metabolism and advanced glycation endproducts (AGEs) such as glyoxal or methylglyoxyl or an enzyme such as lysyl oxidase (LO) enzyme (either in purified form or recombinant), or transglutaminase (Tgase), or a LO or Tgase promotor, or an epoxy or a carbodiimide. Preferably, the crosslinking reagent contains one of the following ranges of agent concentrations or a combination of agent concentrations: at least 0.001% (0.01 mg/ml) of human recombinant transglutaminase, at least 0.01% (0.1 mg/ml) of purified animal liver transglutaminase, at least 0.25% genipin, at least 0.1% proanthrocyanidin, at least 100 mM EDC, at least 100 mM ribose, at least 100 mM L-Threose, at least 50 mM methylglyoxal, at least 50 mM glyoxal, at least 0.001% lysyl oxidase in a 0.1 M urea solution. More than one crosslinking agent may be used.

The crosslinking reagent may include a carrier medium in addition to the crosslinking agent. The crosslinking agent may be dissolved or suspended in the carrier medium to form the crosslinking reagent. In one embodiment, a crosslinking agent is dissolved in a non-cytotoxic and biocompatible carrier medium. The carrier medium is required to be substantially non-cytotoxic in order to mediate the contact of the crosslinking agent to tissues in the living human body without substantial damage to the tissue or surrounding tissue. Preferably, the carrier medium chosen is water, and more preferably, a saline solution. Preferably, the pH of the carrier medium is adjusted to be the same or similar to the tissue environment. Even more preferably, the carrier medium is buffered. In one embodiment of the present invention, the carrier medium is a phosphate buffered saline (PBS).

When the crosslinking agent is dissolved in a carrier medium, the concentration of the crosslinking agent in the carrier medium is not particularly limited. The concentration may be in any amount effective to increase the crosslinking of the tissue while at the same time remaining substantially noncytotoxic.

In accordance with the present invention, the crosslinking reagent is brought into contact with a portion of a native, non-denatured collagenous tissue. As used herein, collagenous tissue is defined to be a structural or load supporting tissue in the body comprised of a substantial amount of collagen. Examples would include intervertebral disc, articular cartilage, fibrocartilage, ligament, tendon, bone, and skin. In general, the portion of the collagenous tissue to be brought into contact with the crosslinking reagent is the portion of the tissue that is subject to loading. Further, where at least some surgical removal of tissue has occurred, the portion of the tissue to be contacted with the crosslinking reagent is at least the portion of the tissue adjacent to the removed tissues. Preferably, the entire remaining or non-surgically altered tissue of a surgically altered joint is contacted with the crosslinking reagent. Further, the tissue adjacent to the surgically altered joint tissues may also be contacted with the crosslinking reagent. In the case of intervertebral joint tissues subjected to posterior bony decompression surgery and discectomy, the tissues to be contacted with the crosslinking reagent would at least include the remaining intervertebral disc.

The collagenous tissues that are particularly susceptible for use in accordance with the present invention include intervertebral discs and fibrocartilage such as knee meniscus. Where the collagenous tissue is an intervertebral disc, the portion of the intervertebral disc that is preferably contacted by the crosslinking reagent is all of the remaining annulus fibrosis. When a collagenous tissue patch is used to block the hole in the disc created or used in the discectomy procedure, the portion of the intervertebral disc that is preferably contacted by the crosslinking reagent is all of the remaining annulus fibrosus, the patch tissue or tissue substitute, and the tissue surrounding the patch. In the case of a partial meniscectomy, or when a partial meniscus tear is removed surgically, the portion of the meniscus that is preferably contacted by the crosslinking reagent is all of the remaining meniscus tissue.

The selected portion of the collagenous tissue must be contacted with an effective amount of the non-toxic crosslinking reagent. An “effective amount” is an amount of crosslinking reagent sufficient to have a mechanical effect on the portion of the tissue treated. Specifically, an “effective amount” of the crosslinking reagent is an amount sufficient to improve the fatigue resistance of the treated tissue, reduce material property degradation resulting from repetitive physiologic loading, or reduce the increase of viscoelastic properties of the treated tissue due to fatigue loading, or reduce the decrease of elastic-plastic properties of the treated tissue due to fatigue loading, or to improve or restore joint stability properties, or reduce bending hysteresis to normal or pre-tissue removal levels, or decrease joint range of motion to normal or pre-tissue removal levels, or decrease neutral zone size to normal or pre-tissue-removal levels, or increase bending elastic energy storage to normal or pre-tissue removal levels. An effective amount may be determined in accordance with the fatigue and degradation resistance testing described herein with respect to Example 1 or in accordance with the stability testing described herein with respect to Example 2.

The method of the present invention includes contacting at least a portion of the collagenous tissue with an effective amount of the crosslinking reagent. The contact may be effected in a number of ways. Preferably, the contacting of collagenous tissue is effected by a means for minimally invasive delivery of the non-cytotoxic crosslinking reagent. Preferably, the contact between the tissue and the crosslinking reagent is effected by injections directly into the select tissue using a needle. Preferably, the contact between the tissue and the crosslinking reagent is effected by injections from a single or minimum number of injection locations. Preferably, an amount of crosslinking solution is injected directly into the targeted tissue using a needle and a syringe. Preferably, a sufficient number of injections are made along the portion of the tissue to be treated so that complete coverage of the portion of the collagenous tissue to be treated is achieved.

Alternatively, contact between the tissue and the crosslinking reagent is effected by placement of a time-release delivery system directly into or onto the target tissue. One time-released delivery system that may be used is a treated membrane or patch. A reagent-containing patch may be rolled into a cylinder and inserted percutaneously through a cannula to the tissue sight, unrolled and using a biological adhesive or resorbable fixation device (sutures or tacks) be attached to the periphery of the targeted tissue.

Another time-released delivery system that may be used is a gel or ointment. A gel or ointment is a degradable, viscous carrier that may be applied to the exterior of the targeted tissue.

Contact also may be effected by soaking or spraying, such as intra-capsular soaking or spraying, in which an amount of crosslinking solutions could be injected into a capsular or synovial pouch.

It should be noted that the methods and compositions treated herein are not required to permanently improve joint stability, or restabilization subsequent to surgical destabilization, and the resistance of collagenous tissues in the human body to mechanical degradation. Assuming that a person experiences 2 to 20 upright, forward flexion bends per day, the improved stability and increased resistance to fatigue associated with contact of the collagenous tissue with the crosslinking reagent, may, over the course of time, decrease. Preferably, however, the improved stability and increased resistance to fatigue lasts for a period of several months to several years without physiologic mechanical degradation. Under such circumstance, the described treatment can be repeated at the time periods sufficient to maintain joint stability and an increased resistance to fatigue resistance. Using the assumption identified above, the contacting may be repeated periodically to maintain the improvement in joint stability and the increased resistance to fatigue. For some treatment, the time between contacting is estimated to correspond to approximately 1 year for some individuals. Therefore, with either a single treatment or with repeated injections/treatments, the method of the present invention improves joint stability and minimizes mechanical degradation of the collagenous tissue over an extended period of time.

Another aspect of the present invention relates to using the aforementioned crosslinking agents as a device or “reagent and application tray” for improving the stabilization of intervertebral discs, for restabilization of surgically destabilized intervertebral discs, for prevention of ongoing joint degradation, for improving the resistance of collagenous tissue to mechanical degradation.

The “reagent and application tray” is sterile and contained within a sterile package. All of the necessary and appropriate and pre-measured reagents, solvents and disposable delivery devices are packaged together in an external package that contains a suitable wrapped sterile “reagent and application tray”. This sterile tray containing the reagents, solvents, and delivery devices is contained in a plastic enclosure that is sterile on the inside surface. This tray will be made available separate from the computer hardware and software package needed to suggest appropriate application positions.

### EXAMPLES 1 and 1A

Thirty-three lumbar intervertebral joints were obtained from ten four-month-old calf spines. The intervertebral joints were arbitrarily divided into 3 groups: untreated controls-12 specimens, genipin treatment 1 (G1)-6 specimens, and genipin treatment 2 (G2)-13 specimens. The G1 treatment involved 72 hours of soaking the whole specimen in PBS with a 0.033% concentration-of genipin. Similarly the G2 treatment involved 72 hours of soaking whole specimens in PBS with 0.33% concentration of genipin. 0.33% Genipin in PBS is produced by dilution of 50 ml of 10 times. PBS (Phosphate Buffered Saline) with distilled water by a factor of 10 to give 500 ml (500 gm) of PBS and mixing in 1.65 grams of genipin to produce the 0.33% (wt %, gm/gm) solution. Previous testing with pericardium and tendon tissue samples demonstrated the reduction of tissue swelling (osmotic influx of water into the tissue) resulting from crosslinking the tissue. Some controls were not subjected to soaking prior to fatigue testing. Others were soaked in a saline solution for 72 hours. Water mass loss experiments were conducted to establish the equivalency of outer annulus hydration between the genipin soaked and 0.9% saline soaked controls. The selection of treatments was randomized by spine and level. The vertebral ends of the specimens were then potted in polyurethane to facilitate mechanical testing.

Indentation testing and compression/flexion fatigue cycling were carried out in the sequence presented in Table 1.

TABLE 1
Experimental protocol

| Measurement | | |
|---|---|---|
| Sequence | Measurement | Location |
| 1 | Stress Relaxation | Center of the Posterior Annulus |
| 2 | Hardness | Center of the Posterior Annulus |

3000 Compression/Flexion Fatigue Cycles

| | | |
|---|---|---|
| 3 | Stress Relaxation | 4 mm Lateral to Center |
| 4 | Hardness | Center of the Posterior Annulus |

Additional 3000 Compression/Flexion Fatigue cycles

| | | |
|---|---|---|
| 5 | Stress Relaxation | 4 mm Lateral to Center |
| 6 | Hardness | Center of the Posterior Annulus |

At the prescribed points in the loading regimen, indentation testing was used to find viscoelastic properties as follows. Stress relaxation data was gathered by ramp loading the 3 mm diameter hemi-spherical indenter to 10 N and subsequently holding that displacement for 60 s, while recording the resulting decrease in stress, referred to as the stress relaxation. Identation testing was also utilized to determine elastic-plastic properties by calculating a hardness index (resistance to indentation) from ramp loading data. Prior to recording hardness measurements, the tissue is repeatedly indented 10 times (60 s/cycle, to the displacement at an initial 10 N load).

This test protocol is based on two principles. First, viscoelastic effects asymptotically decrease with repeated loading. Secondly, hardness measurements are sensitive loading history of the tissue. However this effect becomes negligible following 10 loading cycles. In order to minimize these effects, viscoelastic data (stress relaxation) was collected from tissue that had not previously been indented. Alternately, elastic-plastic data (hardness) was collected from tissue that had been repeatedly loaded (preconditioned). In this case, repetitive indentation was intended to reduce the undesired effects of the changing viscoelastic properties, namely lack of repeatability, on hardness measurements. These testing procedures were derived from several preliminary experiments on the repeatability of the measurements with variations of loading history and location.

Following initial indentation testing, the specimen was loaded repetitively in flexion-compression at 200 N for 3000 cycles at a rate of 0.25 Hz. The load was applied perpendicularly to the transverse plane, 40 mm anterior to the mid-point of the specimen in the transverse plane. A second set of indentation testing data is then collected following fatigue cycling. This procedure was followed for two fatigue loading cycles. During all testing, the specimens were wrapped in saline wetted gauze to maintain their moisture content. Fatigue cycling and non-destructive indentation testing were carried out on an MTS 858.02 biaxial, table-top, 10 kN capacity servo-hydraulic materials test station (MTS, Eden Prairie, Minn.), with the MTS Test Star data acquisition system. Several statistical measures were calculated to evaluate the significance of the results. A nested two-way analysis of variance (ANOVA) was utilized to confirm effects due to treatment and number of fatigue cycles. Due to the non-parametric nature of the data, the Mann-Whitney non-parametric rank-sum test was used to assess the null hypotheses that the treatment did not affect: 1) the pre-cycling mechanical parameters of the tissue, or 2) the amount of change (degradation) in elastic-plastic and viscoelastic mechanical parameters due to fatigue loading. The confidence level for statistical significance was set at p<0.05.

Nested two-way ANOVA analysis determined that both viscoelastic (relaxation) and elastic-plastic (hardness) mechanical parameters were independently affected by fatigue cycling and by treatment type. These statistical results are presented in Table 2.

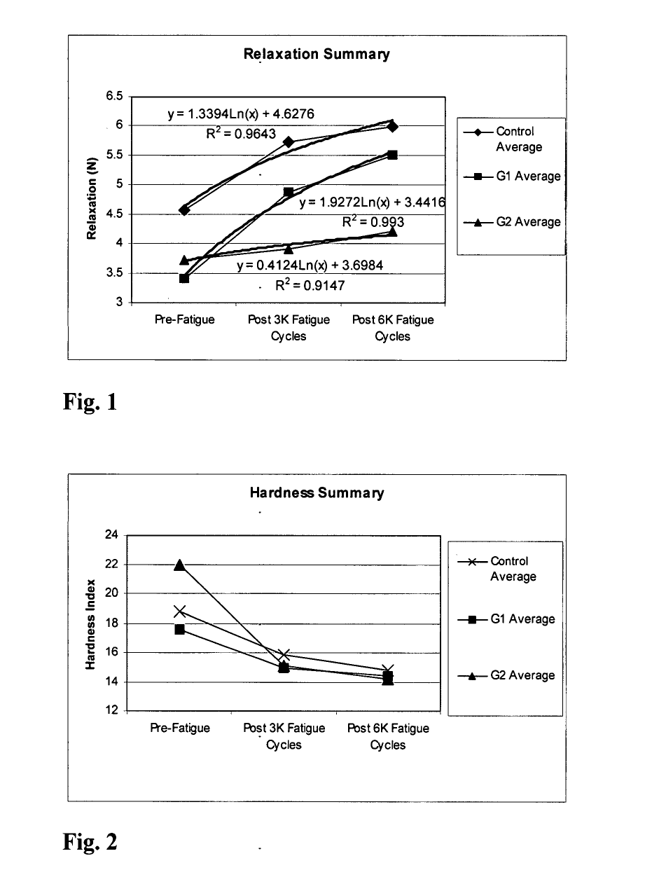

The relaxation test results are presented graphically in FIG. 1. There was an initial shift downward of the relaxation curve caused by the crosslinking treatment. This would represent a beneficial effect as higher stress relaxation would be associated with more severely degraded tissue (Lee 1989). The initial pre-fatigue relaxation of the G1 and G2 treatment groups were 26% and 19% less than (p=0.009 and p=0.026) the pre-fatigue relaxation of the controls respectively. There was also dramatic improvement in fatigue resistance as demonstrated by the change in relaxation after 6000 non-traumatic loading cycles. The change in relaxation due to 6000 fatigue cycles for the G2 treated discs was less than a third of the change in the controls (p=0.044). However, the lesser concentration of Genepin did not bring about the same improvement in fatigue resistance.

The hardness test results are presented graphically in FIG. 2. There is an initial shift upward of the hardness data caused by the G2 crosslinking treatment. This would represent a beneficial effect as loss of hardness would signal a loss of structural integrity in the tissue. The initial pre-fatigue hardness of the G2 treatment group was 17% greater than that of the control group (p=0.026). However this beneficial effect appears to have eroded prior to 3000 fatigue cycles and the change in hardness between 3000 and 6000 cycles is essentially the same for the two groups (G2=0.94, Control=−1.01).

TABLE 2
Results of nested two-way ANOVA analysis

| Material Property | Factor | F-Value | Probability |
|---|---|---|---|
| Stress Relaxation | Treatment | 16.060 | 1.085E−06 |
| | Fatigue Cycling | 9.676 | 2.500E−03 |
| | Interaction | 1.402 | 2.515E−01 |
| Hardness | Treatment | 20.023 | 6.405E−08 |
| | Fatigue Cycling | 5.898 | 1.710E−02 |
| | Interaction | 4.228 | 1.760E−02 |

The data presented above quantifies the elastic and viscoelastic mechanical degradation of intervertebral disc tissue due to repetitive, non-traumatic loading. The results of these experiments establish that non-toxic crosslinking reagents reduce the fatigue-related degradation of material properties in a collagenous tissue—namely the intervertebral disc. More than a three-fold reduction in viscoelastic degradation was brought about by soaking the calf disc tissue in 0.33 g/mol concentration of genipin. The tested formulation was unable to sustain an improvement in the elastic mechanical properties (hardness) to 3000 test cycles.

Accurately estimating the length of time it would take an average person to experience a comparable amount of wear and tear on their spinal discs is difficult. Certainly, in addition to the mechanical degradation imposed by the described testing, there is the added—“natural”—degradation of these dead tissues due to the testing environment. The non-loaded controls showed this “natural” degradation of material properties to be insignificant. Measures were taken to minimize this natural degradation by keeping the specimens moist throughout the testing and by accelerating the loading frequency. At the same time, loading frequency was kept within physiologic limits to prevent tissue overheating. It should be noted that these measures constitute standard protocol for in vitro mechanical testing of cadaveric tissues. Assuming that a person experiences 2 to 20 upright, forward flexion bends per day, these data roughly correspond to several months to several years of physiologic mechanical degradation.

The described treatment could be repeated at the time periods represented by, for instance, 3000 fatigue cycles at this load magnitude. Using the assumption identified above, this number of cycles may be estimated to correspond to approximately 1 year for some individuals. Therefore, with either a single treatment or with repeated injections/treatments, an individual may be able to minimize mechanical degradation of their intervertebral discs over an extended period of time. Another option would involve a time-release delivery system such as a directly applied treated patch, a gel or ointment.

### EXAMPLE 2

While the overall success rate of lumbar discectomy is favorable, biomechanical investigation (Goel, 1985, 1986) and long-term clinical results (Kotilainen, 1993, 1994, 1998) suggest altered kinematic behavior and degenerative changes to the lumbar spine associated with significant loss of nucleus material and disc height, including the potential for lumbar instability. Currently, no treatments are available to aide in the prevention of instability and the subsequent degeneration following disc surgery. However, collagen crosslinking has shown favorable effects on disc tissue, including the ability to resist spinal deformity, and increase tensile strength and nutrient delivery. Therefore, the purpose of this experiment is to demonstrate that exogenous collagen crosslinking following posterior decompression surgery results in enhanced biomechanical properties of the intervertebral joint constituting a restabilization of the joint.

Fifteen fresh-frozen bovine lumbar functional spinal units were used for the experimental protocol utilizing a repeated measures design. An eight-axes materials testing device (EnduraTEC, Minnetonka, Minn.) was used to measure flexibility for each specimen in 3 conditions: intact, post-discectomy, and following collagen crosslinking injections. Following testing of the post-discectomy joints, specimens were separated into two groups based on crosslinker type. Discs were treated with either a non-enzymatic crosslinker (400 mM Methylglyoxal in 1×PBS, n=7) or an organic crosslinker (0.33% genipin in 1×PBS, n=8). The injection treatment consisted of injecting the post discectomy annulus fibrosus with less than 20 cc at 4 locations (directly anterior, directly posterior, and bilateral posterolateral,) using a 21-gauge needle, providing sufficient coverage of the disc. In order for the collagenous intervertebral disc to become adequately crosslinked, specimens remained at room temperature for a period of 48 hours, and were intermittently hydrated with % EDTA solution to prevent biological breakdown of tissue.

Continuous cycles of flexion/extension (sagittal plane) loads (±4 Nm) were applied and consequent motion characteristics were measured. The fourth loading cycle of each condition was used to assess instability. Instability was quantified by calculating Neutral Zone (NZ), % Hysteresis (HYS), Range of Motion (ROM), and % Strain Energy (SE, SE=100−HYS). Variables were normalized with respect to intact values. Pairwise comparisons were made using the Wilcoxon Signed-Rank test (significance level, p≦0.05).

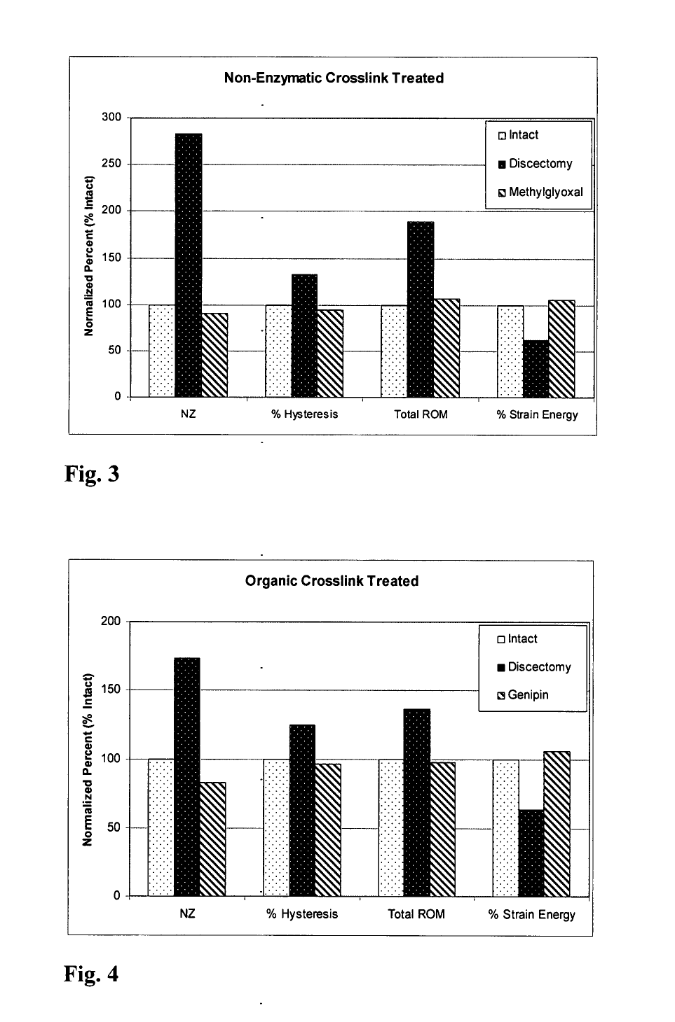

Referring to FIGS. 3 and 4, discectomy induced significant changes in NZ (p=0.009), HYS (p=0.004), ROM (p=0.003), and SE (p=0.004) when compared to intact, demonstrating the destabilizing effect of partial disc removal. All specimens, regardless of crosslinking reagent, showed decreased instability following injection treatment for all variables (all p-values≦0.018). No significant differences existed between intact and post-injection conditions for either group.

Exogenous collagen crosslinking of the intervertebral disc following a common surgical procedure is effective in restabilizing the intervertebral joint in all measured parameters. In fact, under the applied loads used in this study, nonenzymatic (methylglyoxal) and organic (genipin) crosslinking essentially returned each segment to the intact state (most within 6%, NZ within 18%). Implementing exogenous collagen crosslinking as an adjunct to current clinical procedures may be beneficial in preventing or delaying subsequent spinal instability and degenerative change associated with spinal decompression surgery.

One can treat a patient who has undergone posterior decompression surgery including bilateral laminectomies and discectomy by treating the remaining intervertebral disc (annulus fibrosus) at the affected level with a crosslinking agent, such as 400 mM L-Threose in saline (0.15M) or a solution comprised of 200 mM methylglyoxal in saline or a solution of 200 mM glyoxal or a solution 200 mM EDC or a solution comprised of 50-100 μg lysyl oxidase in a 0.1 M urea saline solution or a solution comprised of 50 μg/ml human recombinant transglutaminase in saline, or a solution comprised of 200 μg/ml of purified animal liver transglutaminase in saline. Immediately after the posterior decompression surgery including discectomy or within a few days after surgery the crosslinking agent can be injected into the whole remaining disc at the surgically decompressed levels. According to the preference of the physician administering the treatment, multiple injections of a preferred, non-toxic crosslinking agent can be performed through a single or multiple injection sites. Fluoroscopic or other imaging means can be used to deliver the crosslinking agent to the selected tissues. The patient should be instructed to avoid strenuous activities for a period of a few days.

The invention has been described in terms of certain preferred and alternate embodiments which are representative of only some of the various ways in which the basic concepts of the invention may be implemented. Certain modification or variations on the implementation of the inventive concepts which may occur to those of ordinary skill in the art are within the scope of the invention and equivalents, as defined by the accompanying claims.

### LIST OF REFERENCES

The following publications are hereby incorporated by reference:

Adams, M A, Hutton, W C, Prolapsed intervertebral disc—a hyperflexion injury, Spine, 7:184-191, 1982

Buckwalter, J A, Woo, S L-Y, Goldberg, V M, Hadley, B C, Booth, F, Oegema, T R, Eyre, D R, Current concepts review. Soft-tissue aging and musculoskeletal function, Journal Bone Joint Surgery, 75A:1533-1548, 1993

Buckwalter, J A, Aging and degeneration of the human intervertebral disc, Spine, 20:1307-14, 1995.

Chachra, D, Gratzer, P F, Pereira, C A, Lee, J M, Effect of applied uniaxial stress on rate and mechanical effects of cross-linking in tissue-derived biomaterials, Biomaterials, 17:1865-75, 1996.

Chen, A C, Temple, M M, Ng, D M, Richardson, C D, DeGroot, J, Verzijl, N, teKoppele, J M, Sah, R L, Age-related crosslinking alters tensile properties of articular cartilage, 47.sup.th Annual Meeting, Orthopaedic Research Society, p. 128, 2001.

Chuang and Hedman, Chuang, S-Y, Lin, L-C, Wu, S-S, Popovich, J, Hedman, T

The Biomechanical Properties and Stability of Lumbar Motion Segments Following Posterior Decompression Surgery

Orthopaedic Trans. 32:1025, 2007.

Deutman, R, The case of chemonucleolysis in discogenic sciatica. A review., Acta Orthoaedica Scandinavia, 63:571-575, 1992

Duance, V C, Crean, J K G, Sims, T J, Avery, N, Smith, S, Menage, J, Eisenstein, S M, and Roberts, S, Changes in collagen cross-linking in degenerative disc disease and scoliosis, Spine, 23:2545-51, 1998.

Goel, V K, Goyal S, Clark C, Nishivama K, Nye T. Kinematics of the whole lumbar spine. Effect of discectomy.Spine. 10(6):543-54, 1985.

Goel, V K, Nishiyama, K, Weinstein, J N, Liu, Y K, Mechanical properties of lumbar spinal motion segments as affected by partial disc removal. Spine. 11(10):1008-121986.

Goel, V K, Monroe, B T, Gilbertson, L G, Brinckmann, P, Interlaminar shear stresses and laminae separation in a disc, Spine, 20:689-698, 1995

Green, T P, Adams, M A, Dolan, P, Tensile properties of the annulus fibrosus. II. Ultimate tensile strength and fatigue life, European Spine Journal, 2:209-214, 19931.

Greve, C, Opsahl, W, Reiser, K, Abbott, U, Kenney, C, Benson, D, and Rucker, R, Collagen crosslinking and cartilage glycosaminoglycan composition in normal and scoliotic chickens, Biochemica et Biophysica Acta, 967:275-283, 1988.

Hedman, T. P., Liao, W. L., Yu, J., Watkins, R., Liker, M., Strength Reduction of the Posterior Intervertebral Disc Resulting from Repetitive Sub-Failure Loading, Orthop. Trans., 24: 1017, 1999,

Hedman, T. P., Gray, D., Quantification of intervertebral disc degradation resulting from fatigue, Orthop. Trans., 27: 0120, 2002.

Kotilainen, E, Valtonen, S, Clinical instability of the lumbar spine after microdiscectomy, Acta Neurochir (Wien), 125:120-126, 1993

Kotilainen, E, Valtonen, S, Percutaneous nucleotomy in the treatment of lumbar disc herniation results after a mean follow-up of 2 years, Acta Neurochir (Wien).128(1-4):47-52, 1994,

Kotilainen, E, Long-term outcome of patients suffering from clinical instability after microsurgical treatment of lumbar disc herniation, Acta Neurochir (Wien), 140(2):120-5, 1998.

Lee, J M, Haberer, S A, Boughner, D R, The bovine pericardial xenograft: I. Effect of fixation in aldehydes without constraint on the tensile viscoelastic properties of bovine pericardium, Journal of Biomedical Materials Research, 23:457-475, 1989.

Osti, O L, Vernon-Roberts, B, Moore, R, Fraser, R D, Annular tears and disc degeneration in the lumbar spine: a post-mortem study of 135 discs, J. Bone and Joint Surgery, 74B:678-682, 1992

Panjabi, M M, Yoldas, E, Oxiand, T R, Crisco, J J, Subfailure injury of the rabbit anterior cruciate ligament, J. Orthopaedic Research, 14:216-222, 1996.

Sung H W. Chang Y. Chiu C T. Chen C N. Liang H C. Mechanical properties of a porcine aortic valve fixed with a naturally occurring crosslinking agent. Biomaterials. 20(19): 1759-72, 1999, (a)

Sung, H W, Chang, Y, Chiu, C T, Chen, C N, Liang, H C, Crosslinking characteristics and mechanical properties of a bovine pericardium fixed with a naturally occurring crosslinking agent, Journal Biomed Materials Res., 47:116-126, 1999, (b)

Thompson, J B, Kindt, J H, Drake, B, Hansma, H G, Morse, D E, and Hansma, P K, Bone indentation recovery time correlates with bond reforming time, Nature, 414:773-6, 2001.

Wang, X D, Masilamani, N S, Mabrey, J D, Alder, M E, Agrawal, C M, Changes in the fracture toughness of bone may not be reflected in its mineral density, porosity, and tensile properties, Bone, 23:67-72, 1998.

Zeeman R. Dijkstra P J. van Wachem P B. van Luyn M J. Hendriks M. Cahalan P T. Feijen J. Crosslinking and modification of dermal sheep collagen using 1,4-butanediol diglycidyl ether. Journal of Biomedical Materials Research. 46(3):424-33, 1999.

1. A method of improving the stabilization of an intervertebral joint of a spine subject to elevated collagenous tissue stress as a result of tissue removing surgical decompression surgery, comprising the step of: contacting at least a portion of remaining collagenous tissue within the intervertebral discs with an effective amount of a crosslinking reagent.

2. The method of claim 1 whereby the material properties of the spine are improved relative to restoring or increasing intervertebral joint stability, including one or more of (a) reduced bending hysteresis to normal or pre-tissue removal levels, (b) decreasing joint range of motion to normal or pre-tissue removal levels, (c) decreased neutral zone size to normal or pre-tissue-removal levels, or (d) increased bending elastic energy storage to normal or pre-tissue removal levels.

3. The method of claim 1 wherein the crosslinking reagent contacts at least a portion of the disc.

4. The method of claim 1 wherein the crosslinking reagent is selected from the group consisting of genipin, proanthocyanidin, ribose, threose, glyoxyl, methylglyxol, lysyl oxidase. transglutaminase, a lysyl oxidase promoter, a Tgase promoter, an epoxy, and a carbodiimide.

5. The method of claim 1 further comprising contacting at least a portion of a collagenous tissue within the tissues adjacent to the disc with an effective amount of a crosslinking reagent.

6. The method of claim 1 wherein the contact between the collagenous tissue and the crosslinking reagent is effected by placement of a time-release delivery system directly into or onto the portion of the collagenous tissue.

7. The method of claim 1 further comprising using three-dimensional reconstructions of the collagenous tissue to determine where to contact the collagenous tissue with the crosslinking reagent.

8. The method of claim 1 wherein the contact between the collagenous tissue and the crosslinking reagent is effected by injections directly into the portion of the collagenous tissue with a needle.
